(19) United States
(12) Patent Application Publication
(10) Pub. No.: US 2007/0280994 A1
(43) Pub. Date: Dec. 6, 2007

(54) Ocular Tissue Separation Areas With Barrier Regions For Inlays Or Other Refractive Procedures

(76) Inventor: Crystal M. Cunanan, Mission Viejo, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 11/421,597

(22) Filed: Jun. 1, 2006

(51) Int. Cl. A61F 2/16

(52) U.S. Cl. 424/427; 623/6.11

(57) ABSTRACT

Ocular tissue separation areas having a barrier region are described herein. In one embodiment, the tissue separation area can have an implant placement site configured to accept an ocular implant. The barrier region can be located about at least a portion of the periphery of the implant placement site. The barrier region can include one or more barrier structures. An example barrier structure is a channel formed on one side of the tissue separation area with a corresponding ridge formed on the opposite side. The channel/ridge structure can be configured to impede epithelial ingrowth to the implant placement site.

### FIELD OF THE INVENTION

The field of the invention relates generally to ocular tissue separation areas with barrier regions and, more particularly, to methods and systems for forming intrastromal tissue separation areas with one or more barrier structures located around a lens implantation site.

### BACKGROUND INFORMATION

As is well known, abnormalities in the human eye can lead to vision impairment. Some typical abnormalities include variations in the shape of the eye, which can lead to myopia (near-sightedness), hyperopia (far-sightedness) and astigmatism as well as variations in the tissue present throughout the eye, such as a reduction in the elasticity of the lens, which can lead to presbyopia. Certain devices, generally referred to as implantable lenses, have been used to successfully treat these and other types of vision impairment.

Implantable lenses typically fall into one of two categories: intraocular lenses (IOLs), which may be implanted deep within the eye to replace the eye's natural crystalline lens, and corneal implants, which are typically implanted near the surface of the eye in the cornea to alter the incident light bound for the eye's natural lens. Corneal implants can be classified as an onlay or an inlay. An onlay is an implant that is placed over the cornea such that the outer layer of the cornea, e.g., the epithelium, can grow over and encompass the implant. An inlay is an implant that is implanted into the cornea, typically within a surgically created intrastromal tissue separation area using, for instance, keratophakia.

As for corneal inlays, placement of the implant within the corneal tissue generates a significant concern with regard to preventing the tissue from adversely reacting to the implant. For instance, epithelial cell migration into the corneal tissue separation area can result in corneal haze and, ultimately, anterior stromal necrosis. Both corneal haze and anterior stromal necrosis are a significant concern and can result in lens explantation.

Accordingly, there is a need to reduce the risk of adverse tissue reactions to the presence of ocular implants such as corneal inlays.

### SUMMARY

Ocular implants provided with one or more barrier regions, methods for forming the same and software for forming the same and methods of configuring the software, are provided in this section by the way of exemplary embodiments. These embodiments are examples only and are not intended to limit the invention.

In one exemplary method of forming an intrastromal tissue separation area configured to receive an implant, the method includes forming a base surface in an eye of a subject, at least a portion of the base surface configured to accommodate placement of an implant and forming a barrier structure around a perimeter of the portion of the base surface configured to accommodate placement of the implant, the barrier structure comprising a first sidewall surface, a second sidewall surface and an intermediate surface located therebetween.

In one exemplary method of implanting an ophthalmological device in an eye, the method includes forming a base surface and a complementary cover portion in an eye of a subject, at least a portion of the base surface configured to accommodate placement of an implant, and forming a barrier structure spaced from and around at least a portion of the perimeter of the portion of the base surface configured to accommodate placement of the implant. The barrier structure preferably includes an interface region between the base surface and the cover portion, the interface region being sufficiently irregular to impede the migration of epithelial cells toward the device. The interface region can include a first sidewall surface, a second sidewall surface and an intermediate surface located therebetween.

In an exemplary method for creating an intrastromal tissue separation area in the eye of a subject, the method includes separating a first portion of ocular tissue to form an implant placement site, separating a second portion of ocular tissue to form a channel located at least partially around a perimeter of the implant placement site and separating a third portion of ocular tissue to form an opening configured to allow insertion of the implant therethrough.

In one exemplary embodiment, a computer readable medium is configured to store computer executable instructions for performing a method, the method comprising configuring a tissue separation device to form a base surface in an eye of a subject, at least a portion of the base surface configured to accommodate placement of an implant and configuring the tissue separation device to form a barrier structure in the eye, the barrier structure being located at least partially around the portion of the base surface configured to accommodate placement of the implant.

In another exemplary embodiment, an eye of a subject includes a tissue separation area configured to accept a corneal implant. The tissue separation area can include a base surface having an implant placement site. A barrier region can be located around at least a portion of the implant placement site. The barrier region can include a barrier structure. In one exemplary embodiment, the barrier structure has a first sidewall, a second sidewall and an intermediate surface located therebetween. In another exemplary embodiment, the first sidewall and second sidewall are oriented substantially perpendicular to the intermediate surface.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims. It is also intended that the invention not be limited to the details of the example embodiments.

### BRIEF DESCRIPTION OF THE FIGURES

The details of the invention, including fabrication, structure and operation, may be gleaned in part by study of the accompanying figures, in which like reference numerals refer to like segments.

FIG. 1 is a cross-sectional view of an eye of a subject having an exemplary embodiment of an intrastromal tissue separation area formed therein.

FIG. 2A is a perspective view depicting another exemplary embodiment of an intrastromal tissue separation area.

FIG. 2B is a top-down view depicting another exemplary embodiment of the intrastromal tissue separation area.

FIG. 2C is a cross-sectional view taken along line 2C-2C of FIG. 2B depicting the exemplary embodiment of the intrastromal tissue separation area.

FIG. 2D is an enlarged view of region 2D depicted in FIG. 2C.

FIGS. 2E-F are cross-sectional views taken along line 2C-2C of FIG. 2B depicting additional exemplary embodiments of the intrastromal tissue separation area.

FIG. 3A is a top down view depicting another exemplary embodiment of the intrastromal tissue separation area.

FIG. 3B is a cross-section view of this embodiment of the tissue separation area taken along line 3B-3B of FIG. 3A.

FIG. 4A is a cross-sectional view depicting an exemplary embodiment of the intrastromal tissue separation area.

FIGS. 4B-E are top down views depicting additional exemplary embodiments of the intrastromal tissue separation area.

FIGS. 5A-B are cross-sectional views depicting exemplary embodiments of a barrier structure.

FIG. 6A is a top down view depicting another exemplary embodiment of the intrastromal tissue separation area.

FIG. 6B is a cross-sectional view taken along line 6B-6B of FIG. 6A depicting this embodiment of the intrastromal tissue separation area.

FIGS. 7A-D are cross-sectional views depicting additional exemplary embodiments of the barrier structure.

FIG. 7E is a cross-sectional view depicting another exemplary embodiment of the tissue separation area being located partially on an exemplary embodiment of an implant.

FIG. 8 is a flow diagram depicting an exemplary method of forming an exemplary embodiment of the intrastromal tissue separation area.

FIG. 9 is a flow diagram depicting an exemplary method of configuring system software operating on/with a cutting device system.

### DETAILED DESCRIPTION

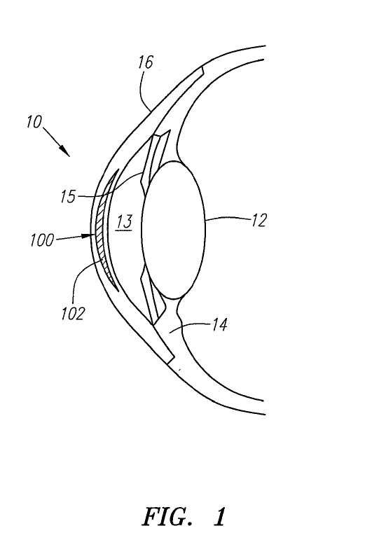

Described herein are methods and systems for forming an ocular tissue separation area (or pocket) having a barrier region. FIG. 1 is a cross-sectional view depicting a human eye 10 in which an exemplary embodiment of an ocular tissue separation area 100 is formed. Labeled within eye 10 is natural lens 12, aqueous humor 13, ciliary bodies 14, iris 15 and cornea 16. For ease of discussion, the methods and systems described herein will be done so in the context of a vision correction procedure done via implantation of a lens 102 into tissue separation area 100 within cornea 16. It should be understood that the methods and systems described can be used in a range of applications broader than that described in the following embodiments. For instance, these applications extend to any type of ocular treatment procedure (e.g., correction of visual impairments such as myopia, hyperopia, presbyopia, excimer corrections and correction of non-visual impairments or defects), at any location within the eye (e.g., intra-corneal or other), and with any type of implant (e.g., implantable lenses, devices or materials and without implants altogether).

Furthermore, tissue separation area 100 will be described as being formed with use of a laser or light-emitting cuffing device (i.e., in a manner similar to those used with Laser in Situ Keratomileusis (LASIK), Laser Epithelial Keratomileusis (LASEK), Photorefractive Keratectomy (PRK), Photoastigmatic Keratectomy (PARK), Holmium Laser ThermoKeratoplasty (LTK), Diode ThermoKeratoplasty (DTK) and the like) although it should be understood that tissue separation area 100 is not limited to formation with solely light-emitting devices. Other manners of formation can be used, including, but not limited to those performed with mechanical cutting devices (e.g., Automated Lamellar Keratoplasty (ALK) performed with a microkeratome, procedures performed with a scalpel and the like), those performed with thermal cutting devices, those performed with any combination of the above and the like.

In this embodiment, tissue separation area 100 is formed intrastromally within cornea 16. Implant 102, which is placed within tissue separation area 100, is preferably a corneal inlay configured to treat a visual impairment. Implant 102 is preferably composed of a material with a permeability sufficient to allow fluid and nutrient transfer between the corneal tissue located anterior and posterior to tissue separation area 100. For instance, in one example embodiment implant 102 is composed of a microporous hydrogel material such as that described in detail in U.S. Pat. No. 6,875,232 entitled “Corneal Implant and Method of Manufacture,” which is fully incorporated by reference herein. Implant 102 can be configured with any shape or form desired to treat the visual impairment. For instance, implant 102 can be disc-like, ring-like, hemispherical, semi-circular, irregular or any other desired shape.

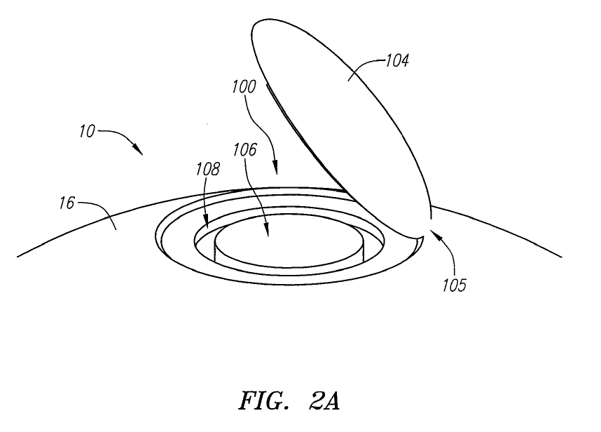

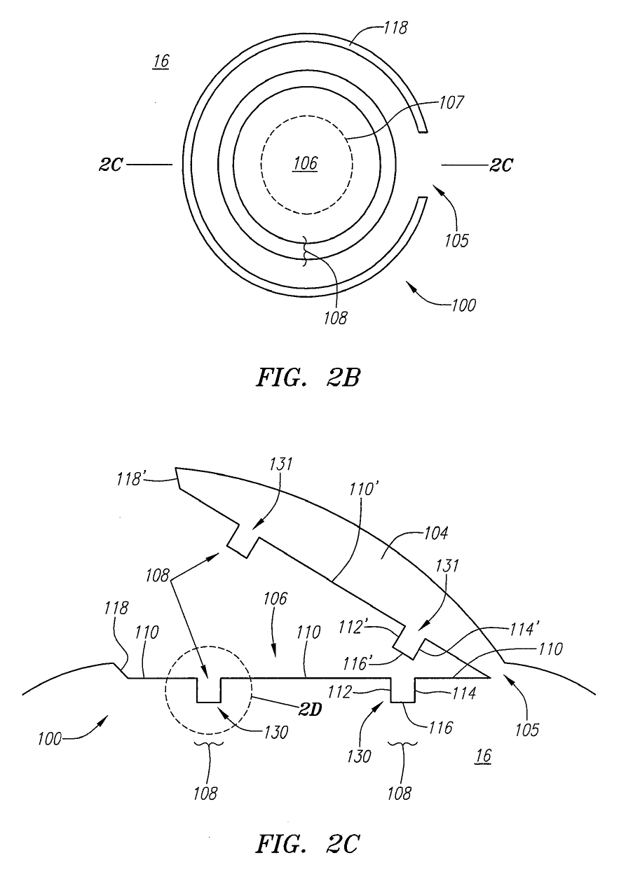

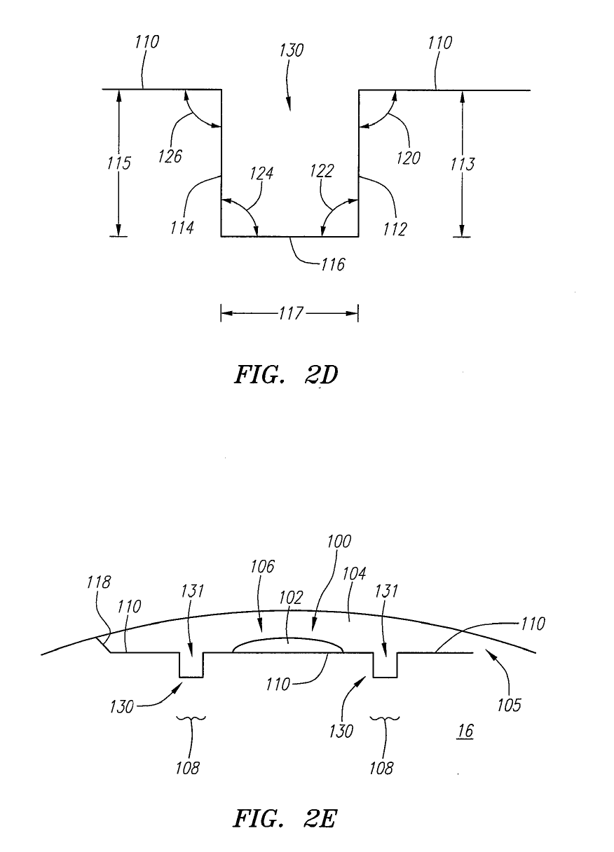

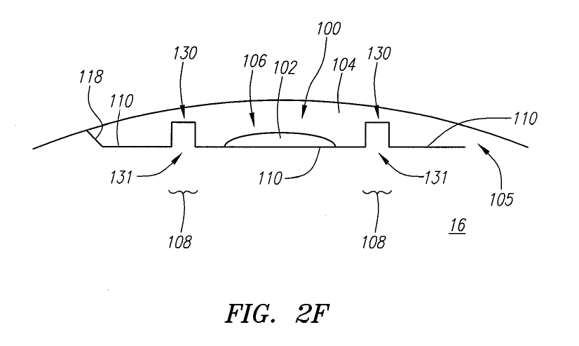

FIG. 2A is a perspective view of an exemplary embodiment of tissue separation area 100. In this embodiment, tissue separation area 100 is formed by separation of a cover portion or flap 104 of corneal tissue from the surface of cornea 16. Tissue separation area 100 is shown here partially open with flap 104 lifted from the surface of cornea 16. A portion of corneal tissue 105 is left connected between flap 104 and cornea 16 to form a hinge about which flap 104 can be opened and closed. FIG. 2B is a top down view of this embodiment in a closed position with flap 104 resting on cornea 16. Base surface 110, which includes an implant placement site 106 and a barrier region 108, also includes the posterior portion of tissue separation area 100. Implant placement site 106 is the desired location on which implant 102 is placed. Barrier region 108 is preferably placed around the perimeter 107 of implant placement site 106, although region 108 can also be placed beneath and/or above implant placement site 106 as well, if desired. Barrier region 108 is the interface between the anterior surface located on the underside of flap 104 and the posterior surface located on base surface 110, which can act as a barrier to the migration of epithelial cells, which is described in more detail below.

FIG. 2C is a cross-sectional view of a plane located within cornea 16 taken along line 2C-2C of FIG. 2B with flap 104 lifted from the surface of cornea 16. It can be seen here that, in this embodiment, a base surface 110 is formed that includes implant placement site 106. Surface 110 is optionally provided with an irregular surface 118 that is spaced from the perimeter 107 of placement site 106 and extends through the stroma and epithelium of the cornea to the exterior to the exterior surface of eye 10. Perimeter surface 118 forms the perimeter of base surface 110 and is preferably sufficiently large to allow insertion of implant 102 to placement site 106.

FIG. 2D is an enlarged view of region 2D depicted in FIG. 2C. FIG. 2D shows a portion of barrier region 108 that includes a barrier structure 130. Barrier structure 130 includes a first sidewall surface 112, a second sidewall surface 114 and an intermediate surface 116 located between sidewall surfaces 112 and 114. Here, surfaces 112, 114 and 116 are together configured such that barrier structure 130 is in the form of a channel positioned posterior to base surface 110. Barrier structure 130, which will be described in more detail below, can be configured to act as a barrier to epithelial migration. For ease of illustration, barrier structure 130 will be referred to as channel 130 in this and subsequent embodiments, although it should be understood that barrier structure 130 is not limited to configuration as a channel.

First sidewall surface 112 is adjacent to base surface 110 and intersects base surface at a first intersection angle 120, which, in this embodiment, is 90 degrees. Likewise, the opposite end of first sidewall surface 112 intersects a first end of intermediate surface 116 at a second intersection angle 122, the opposite end of intermediate surface 116 intersects a first end of second sidewall surface 114 at a third intersection angle 124, and the opposite end of second sidewall surface 114 intersects base surface 110 at a fourth intersection angle 126. Like first intersection angle 120, in this embodiment each of intersection angles 122-126 are also 90 degrees.

As mentioned above, formation of tissue separation area 100 will be described herein as being accomplished by way of a laser or light-emitting cutting device. Formation in this manner is accomplished by separating the corneal tissue such that two surfaces are formed that are complementary to each other. For instance, formation of each of posterior corneal surfaces 110-118 results in the formation of a corresponding complementary surface 110′-118′, respectively, on the underside of flap 104. Each of complementary surfaces 110′-118′ are depicted in FIG. 2C. Because surfaces 110′-116′ are complementary versions of surfaces 110-116, the intersection angles between adjacent surfaces 110′-116′ are substantially the same and are not provided separate reference numerals in FIG. 2C. Surfaces 112′-116′ form a second barrier structure 131, which, in this embodiment, is in the form of a ridge complementary to channel 130. For ease of illustration, barrier structure 131 will be referred to as ridge 131 in this and subsequent embodiments, although it should be understood that barrier structure 131 is not limited to configuration as a ridge.

FIG. 2E is another cross-sectional view of tissue separation area 100 taken along line 2C-2C of FIG. 2B. Here, tissue separation area 100 is shown after the completion of the correction procedure with lens 102 implanted therein. Flap 104 has been brought down to close tissue separation area 100 and ridge 131 is located within channel 130 such that barrier region 108 includes both channel 130 and ridge 131. After completion of the procedure, barrier region 108 acts to inhibit epithelial migration from the outer region of cornea 16 to lens placement site 106. Generally, epithelial cells cannot migrate through corneal tissue and must migrate along a tissue surface instead. However, the cytoskeletal structure of epithelial cells does not allow easy migration across dramatic changes in surface topography. Here, the right angle orientation of surfaces 110-116 provides a topographical barrier to epithelial migration and barrier region 108 can act to inhibit the passage of epithelial cells to lens placement site 106. This can mitigate the risk of corneal haze, anterior stromal necrosis and other undesirable conditions.

To maximize impedance of epithelial migration, barrier region 108 preferably has a plurality of channel-like/ridge-like configurations with intersection angles 120-126 equal to 90 degrees or substantially 90 degrees. As used herein, “substantially 90 degrees” is a broad phrase intended to apply to all instances where a value close to, but different from 90 degrees is actually achieved or intended to be achieved. Variations in corneal tissue, curvature of the eye, the design of tissue separation area 100, tolerance of the cutting device, skill of the physician/technician, and variance during the procedure are just some of the many factors that can result in intersection angles 120-126 being substantially 90 degrees. Also, it should be noted that intersection angles 120-126 can assume values other than 90 degrees. For instance, in one exemplary embodiment, intersection angles 120-126 are each between 75 and 105 degrees, in another exemplary embodiment, intersection angles 120-126 are each between 60 and 120 degrees, in yet another exemplary embodiment, intersection angles 120-126 are each between 45 and 135 degrees, and in still yet another exemplary embodiment, intersection angles 120-126 are each between 30 and 150 degrees.

It should be noted that each intersection angle 120-126 can have a different value from each other. It is not required that each intersection angle have the same value. Also, first sidewall surface 112, second sidewall surface 114 and intermediate surface 116 need not be flat along the cross-sectional plane as depicted in FIGS. 2C-E. Surfaces 112-116 can be curved or otherwise shaped as desired. As depicted in FIG. 2D, first sidewall surface 112 has a length 113, second sidewall surface 114 has a length 115, and intermediate surface 116 has a length 117. Preferably, each surface 112, 114 and 116 has the same length 113, 115 and 117, respectively. In one exemplary embodiment, lengths 113, 115 and 117 are each equal to 20 microns. In another exemplary embodiment, each of lengths 113, 115 and 117 has a value between 10 and 25 microns. It should be noted that lengths 113, 115 and 117 are not limited to this range and can be less than 10 microns or more than 25 microns. Also, like angles 120-126, each of lengths 113, 115 and 117 need not be the same and can in fact be different from each other. Furthermore, barrier region 108 need not be symmetrical. For instance, sidewall 112 can have a length or shape different from sidewall 114 in both the cross-sectional planes depicted in FIGS. 2C-D and otherwise.

The placement of channel 130 and ridge 131 can be reversed from that depicted in FIGS. 2A-E. For instance, surfaces 112, 114 and 116 on the posterior corneal portion of tissue separation area 100 can be configured as ridge 131, while surfaces 112′, 114′ and 116′ on the underside of flap 104 can be configured as channel 130. FIG. 2F is a cross-sectional view depicting an exemplary embodiment of tissue separation area 100 having this reversed configuration (reference numerals for surfaces 112′, 114′ and 116′ are not shown).

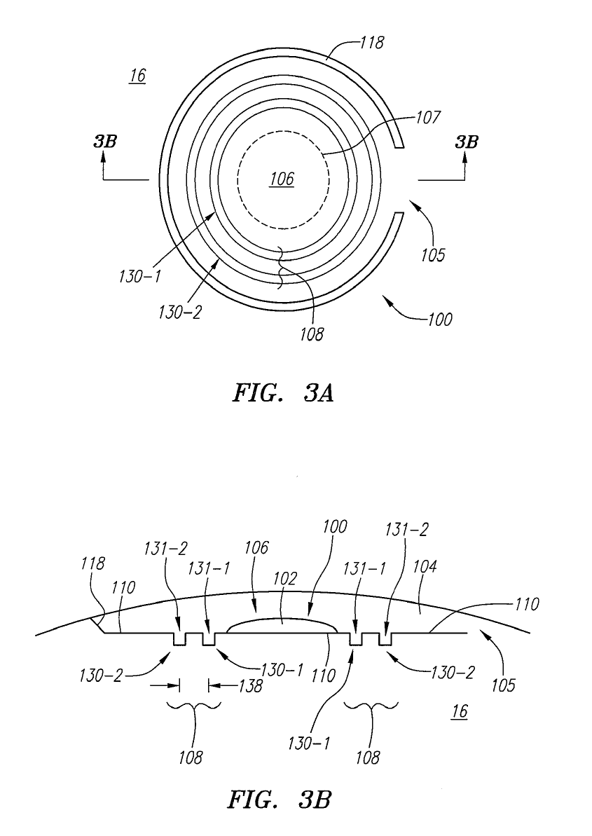

FIG. 3A is a top down view depicting another exemplary embodiment of tissue separation area 100 in a closed state. FIG. 3B is a cross-sectional view of this embodiment of tissue separation area 100 taken along line 3B-3B of FIG. 3A. In this embodiment, barrier region 108 includes two channels 130 with corresponding ridges 131 arranged concentrically around lens placement site 106. The channel 130 and ridge 131 located immediately adjacent to the perimeter of lens placement site 106 is referred to as channel 130-1 and ridge 131-1, while the channel 130 and ridge 131 located further outside lens placement site 106 is referred to as channel 130-2 and ridge 131-2. Generally, when referring to multiple instances of the same or similar structure herein, the notation XXX-Y will be used where XXX is the reference numeral of the structure and Y is the instance of that structure.

In this embodiment, the use of multiple channels 130 (and corresponding ridges 131) provides further protection against epithelial migration, for instance, channel 130-1 can provide another layer of protection in case the structural integrity of channel 130-2 is compromised. Each individual channel 130-1 and 130-2 can be sized, shaped or configured differently to provide an additional types of protection not possible with only one channel 130. Also, in cases where more than two channels 130 are provided, the pitch 138 between each channel 130 can vary as desired.

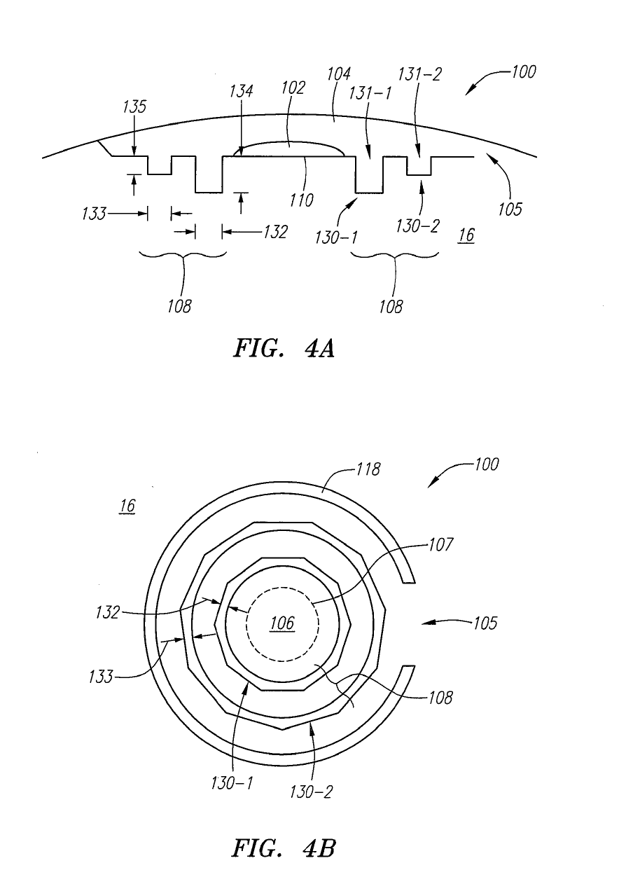

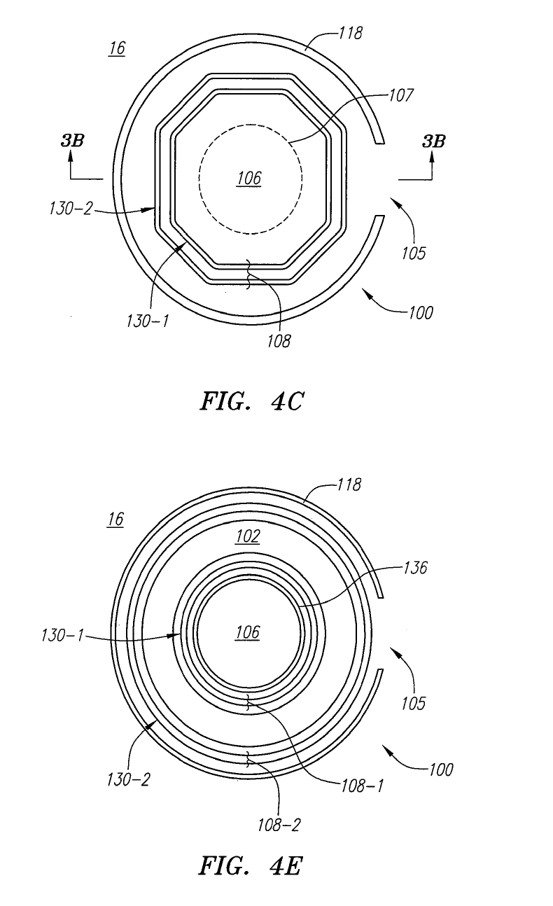

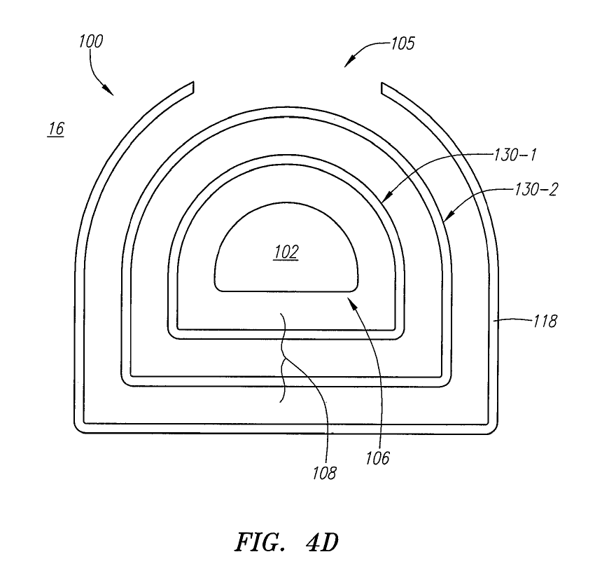

For instance, FIGS. 4A-E depict additional exemplary embodiments of tissue separation area 100 where channels 130-1 and 130-2 are configured with different/variable widths and depths. FIG. 4A is a cross-sectional view depicting an exemplary embodiment where the width 132 and depth 134 of channel 130-1 is greater than the width 133 and depth 135 of channel 130-2. FIG. 4B is a top down view of another exemplary embodiment of tissue separation area 100 where channel 130-1 and 130-2 have varying widths 132 and 133. Although not shown, depths 134 and 135 can vary as well.

FIG. 4C is a top down view depicting another exemplary embodiment of tissue separation area 100 where channels 130-1 and 130-2 have hexagonal shapes with rounded corners. Channel 130 can also be configured based on the shape of implant 102. For instance, FIG. 4D is a top down view depicting an exemplary embodiment of tissue separation area 100 with implant 102 located therein. Here, implant 102, implant placement site 106 and channels 130-1 and 130-2 each have corresponding hemispherical shapes. FIG. 4E is a top down view depicting an exemplary embodiment where implant 102 and implant placement site 106 are both annular. In this embodiment, flap 104 is also annular and a second barrier region 108-2 is placed around the interior of the annular implant 102 to prevent epithelial ingrowth from the inner peripheral surface 136 of tissue separation area 100. It should be noted that each channel 130 can be configured with other shapes, regardless of the shape of implant 102, including, but not limited to other polygons or multi-sided shapes, circles, ovals, ellipses, symmetrical and asymmetrical shapes, irregular shapes and any combination thereof.

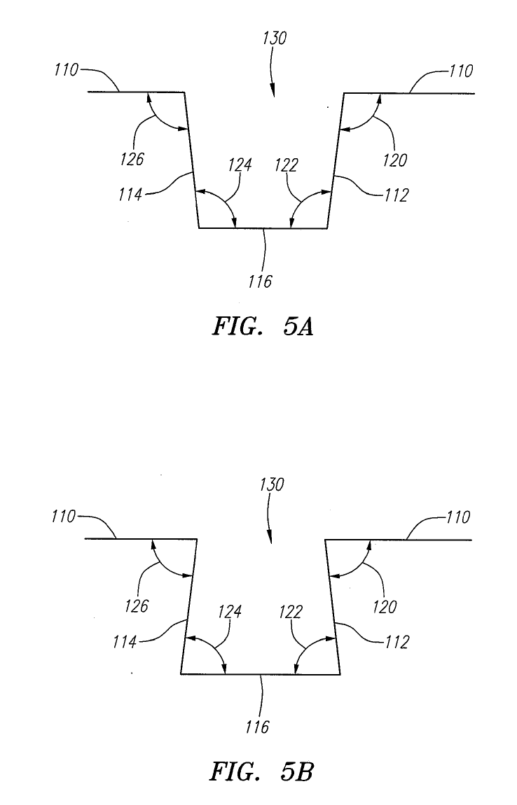

As mentioned above, intersection angles 120-126 can be any angle, preferably substantially 90 degrees, and each angle 120-126 in a given configuration does not have to be the same. FIG. 5A is a cross-sectional view depicting an exemplary embodiment of tissue separation area 100 where each of intersection angles 120-126 are approximately 100 degrees. FIG. 5B is a cross-sectional view depicting an exemplary embodiment of tissue separation area 100 where each of intersection angles 120-126 are approximately 80 degrees. Here, removal of ridge 131 from channel 130 when lifting flap 104 can be more difficult because the posterior portion of ridge 131 is wider than the anterior portion, although the elastic nature of the corneal tissue can allow use of this configuration.

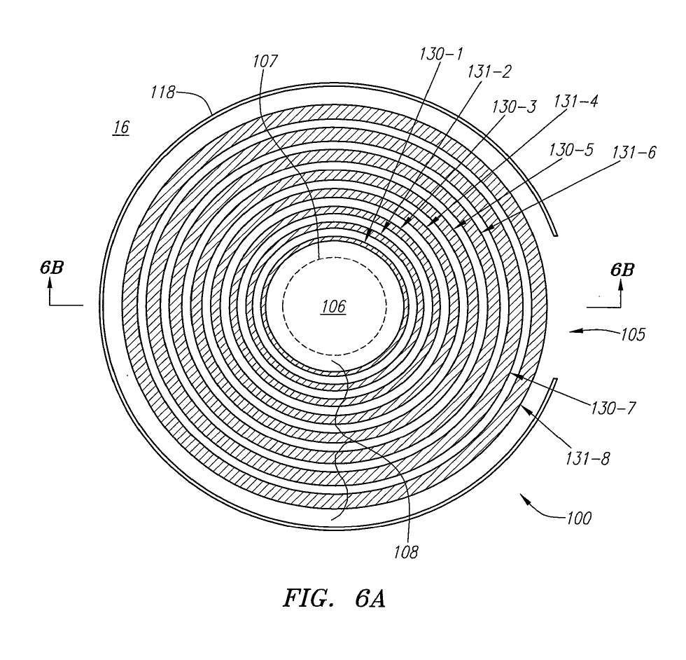

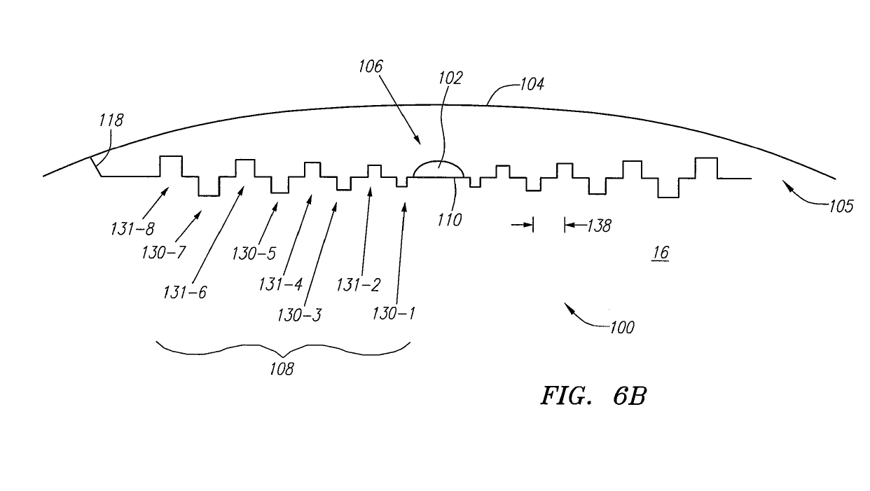

FIG. 6A is a top down view depicting another exemplary embodiment of tissue separation area 100 with flap 104 in the lifted position. FIG. 6B is a cross-sectional view of this embodiment taken along line 6B-6B of FIG. 6A with tissue separation area 100 closed with implant 102 located therein. This embodiment demonstrates just one of the many different ways in which barrier region 108 can be configured. Here, barrier region 108 includes eight pairs of channels 130 and ridges 131 (note that only the channels 130 and ridges 131 for the posterior corneal surface are labeled, the channels 130 and ridges 131 for the underside of flap 104 are not labeled). Surface lengths 113, 115 and 117 (reference numerals not shown) of each channel 130 and ridge 131 progressively increase as the distance from implant placement site 104 grows, although lengths 113, 115 and 117 within each channel remain substantially equal to each other. The pitch 138 (reference numerals not shown) between adjacent channels also increases with the distance from implant placement site 104. In this embodiment the arrangement of each channel 130 and ridge 131 alternates such that channel 130-1 is formed in main cornea 16 with ridge 131-1 formed in flap 104, channel 130-2 is formed in flap 104 with ridge 130-2 formed in cornea 16, channel 130-3 is formed in cornea 16 while ridge 131-3 is formed in flap 104, and so on.

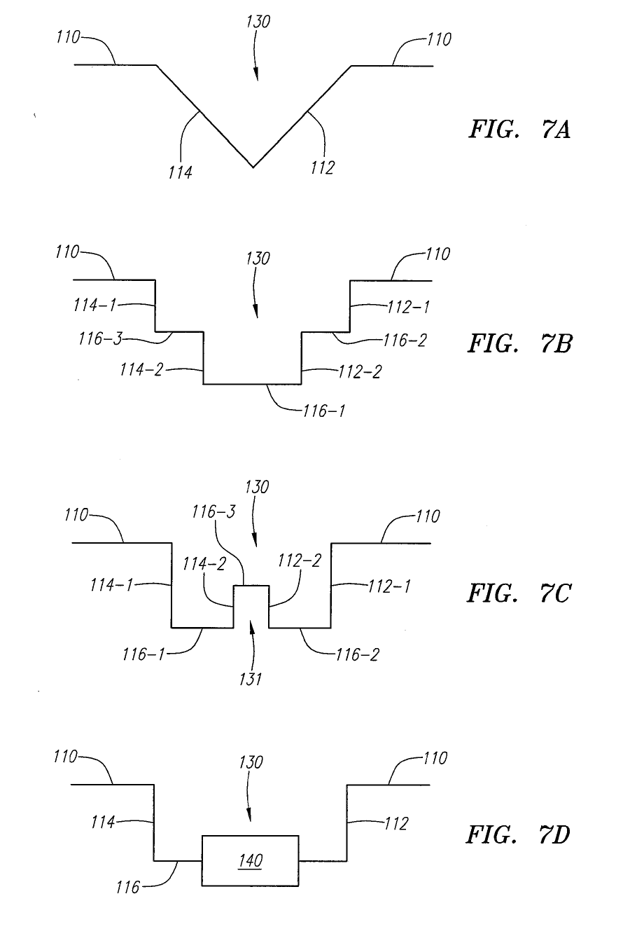

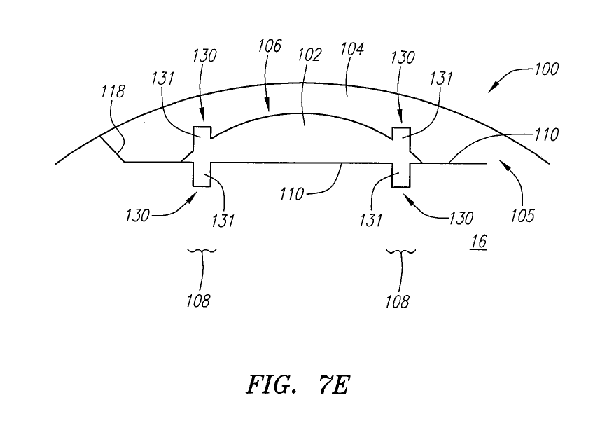

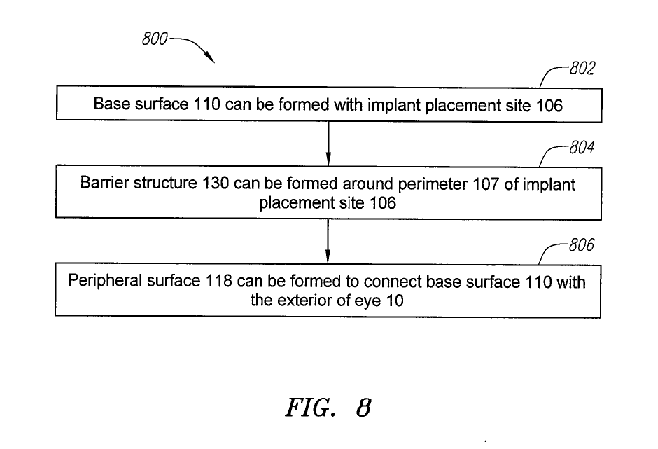

In the above embodiments, each channel 130 has a “box-like” configuration. Channel 130 (and corresponding ridge 131) can have other configurations while at the same time acting as impediments to epithelial migration. FIGS. 7A-D are cross-sectional views depicting just a few of the many additional exemplary embodiments of channels 130 that can be configured for use within barrier region 108. For instance, FIG. 7A depicts an exemplary embodiment where channel 130 has a two-sided saw-tooth configuration with intermediate surface 116 omitted. FIG. 7B depicts an exemplary embodiment where channel 130 has a stepped, multi-channel configuration. An upper portion of channel 130 has two sidewalls 112-1 and 114-2 and a lower portion has two sidewalls 112-2 and 114-2 with intermediate surface 116-1 located therebetween. A second intermediate surface 116-2 is located between sidewalls 112-1 and 112-2 and a third intermediate surface 116-3 is located between sidewalls 114-1 and 114-2. FIG. 7C depicts an exemplary embodiment where channel 130 has two sidewalls 112-1 and 114-1 separated by a first intermediate surface 116-1, a ridge 131 that is relatively smaller than channel 130 and a second intermediate surface 116-2. Ridge 131 itself includes two sidewalls 112-2 and 114-2 and an intermediate surface 116-3 located therebetween. FIG. 7D depicts an exemplary embodiment where channel 130 has a predetermined surface variation 140 along intermediate surface 116 (depicted non-schematically here as a box), which can be used for other purposes relating to tissue separation area 100, the implantation procedure or the treatment procedure.

In each of the embodiments described thus far, channel 130 and ridge 131 are formed in the tissue of the eye. However, channel 130 or ridge 131 can be formed on the surface of implant 102 as well. For instance, FIG. 7E is a cross-sectional view depicting an exemplary embodiment where channels 130 are formed in the underside of flap 104 and the upper surface of cornea 16. Complementary ridges 131 are formed on the top and bottom surfaces of implant 102 and configured to interface with channels 130 as depicted here. It should be noted that any of the variations in the embodiments described with respect to FIGS. 2A-7D can also be applied to this embodiment.

FIG. 8 is a flow diagram depicting an exemplary method 800 for forming an exemplary embodiment of tissue separation area 100. First, at 802, base surface 110 can be formed with implant placement site 106. Next, at 804, barrier structure 130 can be formed around the perimeter 107 of implant placement site 106. Barrier structure 130 can be formed on the posterior cornea 16, on the overlying flap 104, or both. If multiple barrier structures 130 are desired, each can be formed during 804. Then, at 806, a peripheral surface 118 can be formed to connect base surface 110 with the exterior of the eye 10. As mentioned above, the formation of peripheral surface 118 is optional and, in the case it is omitted, base surface 110 can be formed directly to the exterior of the eye 10.

In this embodiment, formation of tissue separation area 100 is described as flowing sequentially from 802 to 806. However, formation does not have to proceed in this order and steps 802, 804 and 806 can be performed in any order desired. As mentioned above, a light-emitting cutting device is preferably used to separate the corneal tissue and thereby form the various surfaces and structures of tissue separation area 100, although method 800 is not limited to such.

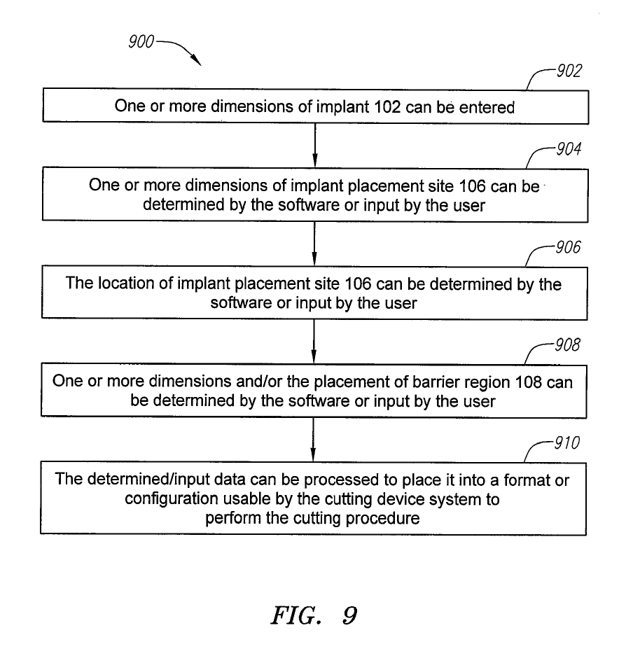

FIG. 9 is a flow diagram depicting an exemplary method 900 of configuring system software operating on, or in communication with, a cutting device system, which can be a light-emitting cutting device used in a LASIK or similar procedure or any other type of cutting device, to form tissue separation area 100. At 902, one or more dimensions of implant 102 can be entered. In an alternative embodiment, the dimensions of implant 102 are determined automatically by the software based on information regarding the particular ocular condition to be corrected. At 904, one or more dimensions of implant placement site 106 can be determined by the software or input by the user. At 906, the location of implant placement site 106 can be determined by the software or input by the user. At 908, one or more dimensions and/or the placement of barrier region 108 can be determined by the software or input by the user. This can be performed prior to determination/input of the location and/or dimensions of implant placement site 106. Portion 908 can also be performed automatically by the software. In another embodiment, the dimensions of barrier region 108 can be entered directly by a user.

Optionally, an optimal number of barrier structures 130 (e.g., channels and/or ridges or others) can be determined by the software, or input by the user. The configuration and/or dimensions of each structure 130 can also be determined automatically by the system software or input by the user.

At 910, the determined/input data can be processed to place it into a format or configuration usable by the cutting device system to perform the cutting procedure. Also, the determined/input data can be processed to place it into a format or configuration for display to the user, either by the system software or a separate software application in communication with the system software.

In the foregoing specification, the invention has been described with reference to specific embodiments thereof. It will, however, be evident that various modifications and changes may be made thereto without departing from the broader spirit and scope of the invention. For example, each feature of one embodiment can be mixed and matched with other features shown in other embodiments. As another example, the order of steps of method embodiments may be changed. Features and processes known to those of ordinary skill may similarly be incorporated as desired. Additionally and obviously, features may be added or subtracted as desired. Accordingly, the invention is not to be restricted except in light of the attached claims and their equivalents.

What is claimed is:

1. A method of forming an intrastromal tissue separation area configured to receive an implant, comprising:
forming a base surface in an eye of a subject, the base surface including an implant placement site configured to accommodate placement of an implant; and
forming a plurality of barrier structures in the base surface around a perimeter of the implant placement site, at least one of the barrier structures comprising a first sidewall surface, a second sidewall surface and an intermediate surface located therebetween.

2. The method of claim 1, wherein at least one barrier structure extends entirely around the implant placement site.

3. The method of claim 1, wherein at least one barrier structure is configured to impede the migration of epithelial cells towards the implant placement site.

4. The method of claim 1, wherein formation of the base surface creates an opening configured to allow insertion of the implant therethrough.

5. The method of claim 1, further comprising forming an opening configured to allow insertion of the implant therethrough.

6. The method of claim 1, wherein the implant is an implantable lens.

7. The method of claim 1, wherein the first sidewall surface intersects the intermediate surface at a first intersection angle between 75 and 105 degrees.

8. The method of claim 7, wherein, in at least one barrier structure, a first portion of the first sidewall surface intersects a first portion of the intermediate surface at the first intersection angle, and a second portion of the second sidewall surface intersects a second portion of the intermediate surface at a second intersection angle.

9. The method of claim 7, wherein the first intersection angle is substantially 90 degrees.

10. The method of claim 7, wherein the first intersection angle is 90 degrees.

11. The method of claim 7, wherein the second sidewall surface intersects the intermediate surface at a second intersection angle of between 80 and 100 degrees.

12. The method of claim 11, wherein the first and second intersection angles are substantially 90 degrees.

13. The method of claim 11, wherein the first and second intersection angles are 90 degrees.

14. The method of claim 13, wherein the first sidewall surface, the second sidewall surface and the intermediate surface are configured as a channel.

15. The method of claim 13, wherein the first sidewall surface, the second sidewall surface and the intermediate surface are configured as a ridge.

16. The method of claim 1, wherein each of the barrier structures comprise a first sidewall surface, a second sidewall surface and an intermediate surface.

17. The method of claim 16, wherein the first sidewall surface intersects the first intermediate surface at a first intersection angle, the second sidewall surface intersects the first intermediate surface at a second intersection angle, and wherein each of the first and second intersection angles is between 30 and 150 degrees.

18. The method of claim 17, wherein each of the first and second intersection angles is between 45 and 135 degrees.

19. The method of claim 18, wherein each of the first and second intersection angles is between 60 and 120 degrees.

20. The method of claim 19, wherein each of the first and second intersection angles is between 75 and 105 degrees.

21. The method of claim 20, wherein each of the first and second intersection angles is substantially 90 degrees.

22. The method of claim 19, wherein each of the plurality of barrier structures are configured as a channel.

23. The method of claim 19, wherein each of the plurality of barrier structures are configured as a ridge.

24. The method of claim 19, wherein at least one of the plurality of barrier structures is configured as a channel.

25. The method of claim 1, wherein the barrier structure is adjacent to the perimeter of the implant placement site.

26. The method of claim 1, wherein the barrier structure is spaced apart from the perimeter of the implant placement site.

27. A method of implanting an ophthalmological device in the cornea of an eye, comprising:
forming a tissue separation area in the cornea, said tissue separation area having a base surface with a portion thereof configured to accommodate the device and a cover portion configured to cover the device and to interface with the base surface;
forming at least one barrier region comprising corresponding irregular surface regions on the base surface and the cover portion, the barrier region being spaced from and at least partially surrounding the portion of the base surface configured to accommodate the device;
implanting the device; and
bringing the cover portion into contact with the base surface such that the barrier region comprises an irregular interface between the cover portion and the base surface that is sufficiently irregular to impede the migration of epithelial cells toward the device.

28. The method of claim 27, wherein the irregular surface regions on the base surface are configured as a channel, and wherein the irregular surface regions on the cover portion are configured as a ridge complementary to the channel.

29. The method of claim 27, wherein the irregular surface regions on the base surface are configured as a ridge, and wherein the irregular surface regions on the cover portion are configured as a channel complementary to the ridge.

30. The method of claim 27, wherein the irregular surface regions in the barrier region interface each other at substantially 90 degrees.

31. The method of claim 27, wherein the irregular surface regions in the barrier region interface each other at 90 degrees.
